(19) United States
(12) Patent Application Publication
(10) Pub. No.: US 2019/0175869 A1
(43) Pub. Date: Jun. 13, 2019

(54) MEDICAL DEVICES INCLUDING RING MEMBERS AND CONNECTING MEMBERS

(71) Applicant: BOSTON SCIENTIFIC SCIMED, INC., MAPLE GROVE, MN (US)

(72) Inventors: DAVID CORY KIRT, MINNETONKA, MN (US); MATTHEW NELSON FROST, NEW BRIGHTON, MN (US)

(73) Assignee: BOSTON SCIENTIFIC SCIMED, INC., MAPLE GROVE, MN (US)

(21) Appl. No.: 16/217,032

(22) Filed: Dec. 11, 2018

Related U.S. Application Data
(60) Provisional application No. 62/597,747, filed on Dec. 12, 2017.

(51) Int. Cl. A61M 25/00; B23K 26/00; B23K 26/362

(52) U.S. Cl. CPC A61M 25/0013; A61M 25/0015; A61B 2017/00526; B23K 26/362; B23K 26/0093

(57) ABSTRACT

Medical devices and methods for making and using medical devices are disclosed. An example method for manufacturing a medical device may include cutting a tubular body into a plurality of ring members. The plurality of ring members may include a first ring member and a second ring member. A connecting member may extend between the first ring member and the second ring member. The method may also include disposing a polymeric member along the plurality of ring members to define a tubular member and articulating the tubular member to break the connecting member.

### CROSS-REFERENCE TO RELATED APPLICATIONS

This application claims the benefit of priority under 35 U.S.C. § 119 of U.S. Provisional Application No. 62/597,747, filed Dec. 12, 2017, the entire disclosure of which is hereby incorporated by reference.

### TECHNICAL FIELD

The present disclosure pertains to medical devices, and methods for manufacturing medical devices. More particularly, the present disclosure pertains to medical devices including ring members and connecting members.

### BACKGROUND

A wide variety of intracorporeal medical devices have been developed for medical use, for example, intravascular use. Some of these devices include endoscopes, guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example method for manufacturing a medical device is disclosed. The method comprises: cutting a tubular body into a plurality of ring members, wherein the plurality of ring members includes a first ring member and a second ring member, wherein a connecting member extends between the first ring member and the second ring member; disposing a polymeric member along the plurality of ring members to define a tubular member; and articulating the tubular member to break the connecting member.

Alternatively or additionally to any of the embodiments above, cutting a tubular body into a plurality of ring members includes laser cutting.

Alternatively or additionally to any of the embodiments above, cutting a tubular body into a plurality of ring members includes etching.

Alternatively or additionally to any of the embodiments above, disposing a polymeric member along the plurality of ring members includes disposing the polymeric member along at least a portion of an outer surface of the plurality of ring members.

Alternatively or additionally to any of the embodiments above, disposing a polymeric member along the plurality of ring members includes encapsulating at least a portion of the plurality of ring members within the polymeric member.

Alternatively or additionally to any of the embodiments above, a second connecting member extends between the first ring member and the second ring member, and wherein articulating the tubular member to break the connecting member includes breaking the second connecting member.

Alternatively or additionally to any of the embodiments above, further comprising attaching the tubular member to a shaft member.

Alternatively or additionally to any of the embodiments above, wherein the shaft member includes a braided region, wherein the tubular member includes a proximal ring member with a braid attachment region, and wherein attaching the tubular member to a shaft member includes securing the braid attachment region of the proximal ring member to the braided region.

Alternatively or additionally to any of the embodiments above, further comprising attaching a steering member to the tubular member.

Alternatively or additionally to any of the embodiments above, attaching a steering member to the tubular member includes disposing the steering member along an inner surface of the tubular member and securing the steering member to a distal end region of the tubular member.

A medical device is disclosed. The medical device comprises: a tubular member including a plurality of ring members, wherein the plurality of ring members includes a distal ring member, a first body region ring member, a second body region ring member, and a proximal ring member; a frangible connecting member extending between the first body region ring member and the second body region ring member; a polymeric member disposed along the tubular member; and a shaft member coupled to the proximal ring member.

Alternatively or additionally to any of the embodiments above, further comprising a second frangible connecting member extending between the first body region ring member and the second body region ring member.

Alternatively or additionally to any of the embodiments above, the first body region ring member has a knuckle region and wherein the knuckle region is nested with a valley region of the second body region ring member.

Alternatively or additionally to any of the embodiments above, the shaft member includes a braid and wherein the proximal ring member includes a braid attachment region coupled to the braid.

Alternatively or additionally to any of the embodiments above, further comprising a steering member coupled to the tubular member.

Alternatively or additionally to any of the embodiments above, the steering member extends along an inner surface of the tubular member and is secured to the distal ring member.

Alternatively or additionally to any of the embodiments above, the polymeric member includes a sleeve disposed along an outer surface of the tubular member.

Alternatively or additionally to any of the embodiments above, the polymeric member encapsulates the tubular member.

A method for manufacturing a medical device is disclosed. The method comprises: encapsulating a plurality of interconnected ring members in a polymeric member to form a tubular member, the plurality of interconnected ring members including a distal ring member, a first body region ring member, a second body region ring member, a frangible connecting member extending between the first body region ring member and the second body region ring member, and a proximal ring member; articulating the tubular member to break the frangible connecting member; and coupling the proximal ring member to a shaft member.

Alternatively or additionally to any of the embodiments above, the plurality of interconnected ring members includes a third body region ring member and a second frangible connecting member extending between the second body region ring member and the third body region ring member, and wherein articulating the tubular member to break the frangible connecting member breaks the second frangible connecting member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic side view of an example medical device.

FIG. 2 is a perspective view of an example tubular member.

FIG. 3 is a side view of a portion of an example tubular member.

FIG. 4 is a perspective view of an example ring member.

FIG. 5 is a perspective view of an example ring member.

FIG. 6 is a perspective view of an example ring member.

FIG. 7 is a perspective view of a portion of an example tubular member.

FIG. 8 is a perspective view of an example tubular member.

FIG. 9 is a perspective view of an example tubular member.

FIG. 10 is a side view of a portion of an example tubular member.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Medical devices with steerable distal tip regions may be manipulated by applying compressive or tensile forces on the tip, for example through the use of a steering wire. It may be desirable to structurally support the distal regions so as to minimize kinking and so as to provide good torque transfer along the distal regions. In some instances, the distal region may be supported by a coil or a braid. The use of a coil to support the distal region may provide good kink resistance. However, the coil may not transfer torque very efficiently. The use of a braid to support the distal region may provide good torque transfer. However, the braid may not resist kinking very efficiently. Disclosed herein are medical device that may include a tubular member. The tubular member may be steerable. In addition, the tubular member may provide good kink resistance and good torque transfer. Some additional details regarding some of the contemplated medical devices are disclosed herein.

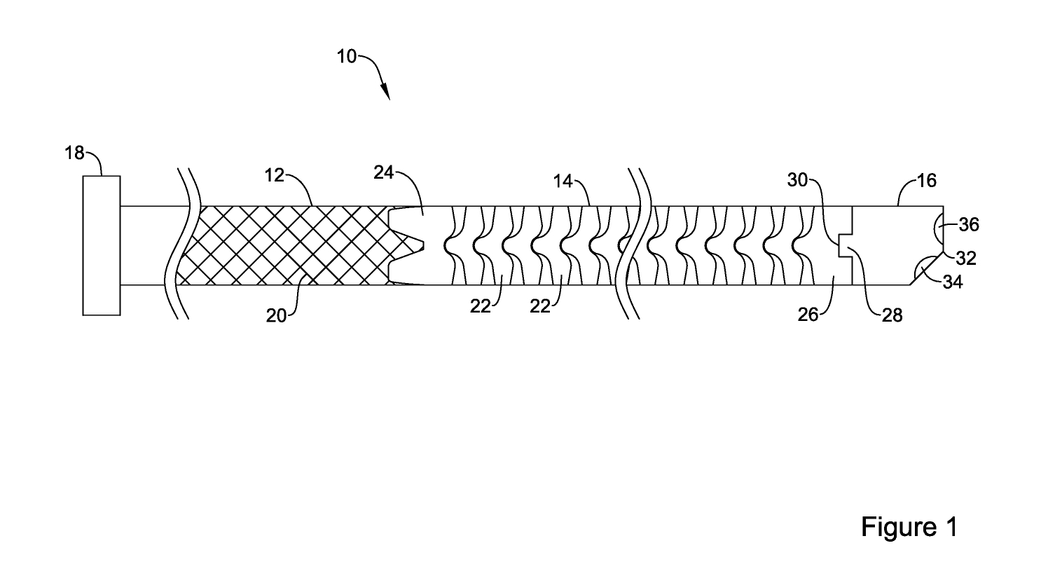

FIG. 1 schematically illustrates a medical device 10. The medical device 10 may include a shaft member 12, a tubular member 14, and a tip member 16. A hub or handle 18 may be coupled to the shaft member 12. In at least some instances, the medical device 10 may take the form of an endoscope, duodenoscope, other scope device, catheter, or the like. When the medical device 10 takes the form of an endoscope (and/or another scope device), the handle 18 may be an endoscope handle having a number of features that may be appropriate for an endoscope handle 18. When the medical device 10 takes the form of a catheter, the hub/handle 18 may take the form of a manifold, hub, adapter, or the like and may include one or more ports/connectors. A number of different configurations are contemplated.

In at least some instances, the shaft member 12 may be considered to be a proximal shaft or region of the medical device (e.g., the proximal shaft or region of an endoscope). The shaft member 12 may include one or more sections or regions (which may include a plurality of continuous regions or a plurality of discrete regions that are secured together). One or more lumens or channels may extend through the shaft member 12. The shaft member 12 may include a support member 20. The support member 20 may take the form of a braid, a coil, a mesh, or the like, or combinations thereof. In at least some instances, the support member 20 may extend along the entire length of the shaft member 12. Alternatively, the support member 20 may extend along one or more portions of the shaft member 12.

The tubular member 14 may include a number of ring members such as the ring members 22. The ring members 22 may be disposed between a first or proximal ring member 24 and a second or distal ring member 26. In some instances, the ring members 22 may be understood to define the body region of the tubular member 14 and, as such, the ring members 22 may be understood to be body region ring members 22. In some instances, all of the ring members 22 may have substantially the same shape, size, configuration, etc. Alternatively, one or more of the ring members 22 may differ for other ring members 22. Some additional details regarding the ring members 22, the proximal ring member 24, and the distal ring member 26 are disclosed herein.

The tip member 16 may be a generally cylindrical member that is coupled to the distal ring member 26. For example, the tip member 16 may include one or more projections 28 designed to engage corresponding cutouts 30 of the distal ring member 26. The shape, size, number, and/or configuration of the projections 28 (as well as the cutouts 30) may vary. In some instances, the tip member 16 may have a substantially flat distal end. Alternatively, a portion of the distal end region of the tip member 16 may be cut at an angle to define an angled distal end region 32. The tip member 16 may include a plurality of openings such as openings 34, 36. The number, size, shape, orientation, or the like of the openings 34, 36 may vary. For example, the tip member 16 may include more or fewer openings. The openings 34, 36 may communicate with one or more lumens formed in the medical device 10 and the openings 34, 36 may allow a number of devices to be positioned at and/or advanced through the distal end of the medical device 10. For example, the openings 34, 36 may allow an endoscopic instrument, imaging device (e.g., a camera), a sensor, guidewire, catheter, or the like to be positioned at and/or advanced through the distal end of the medical device 10.

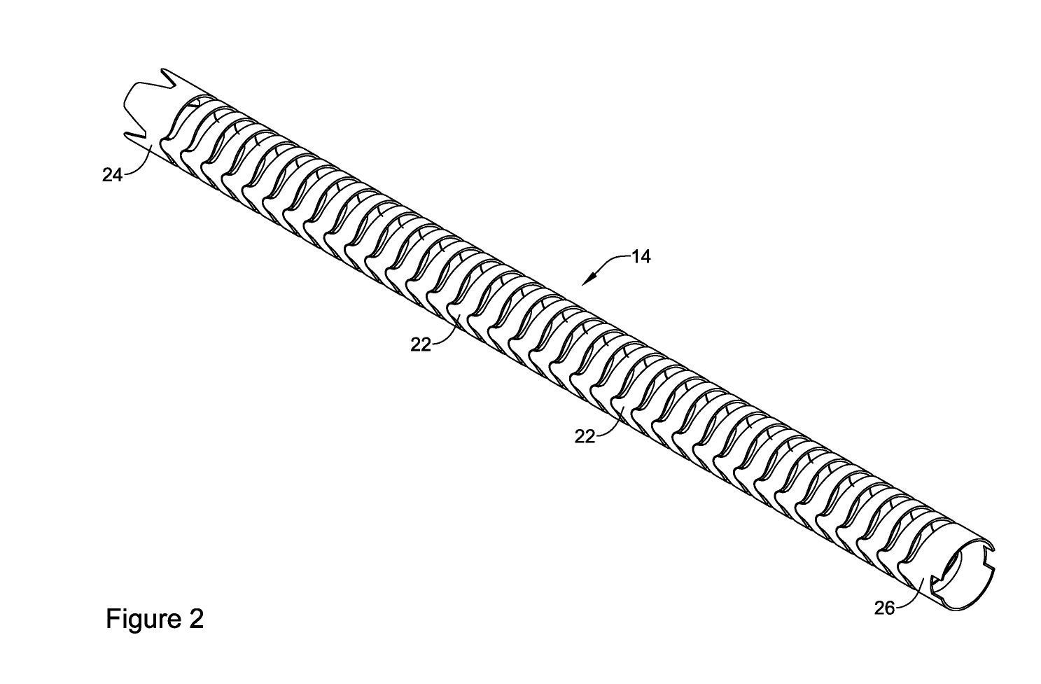

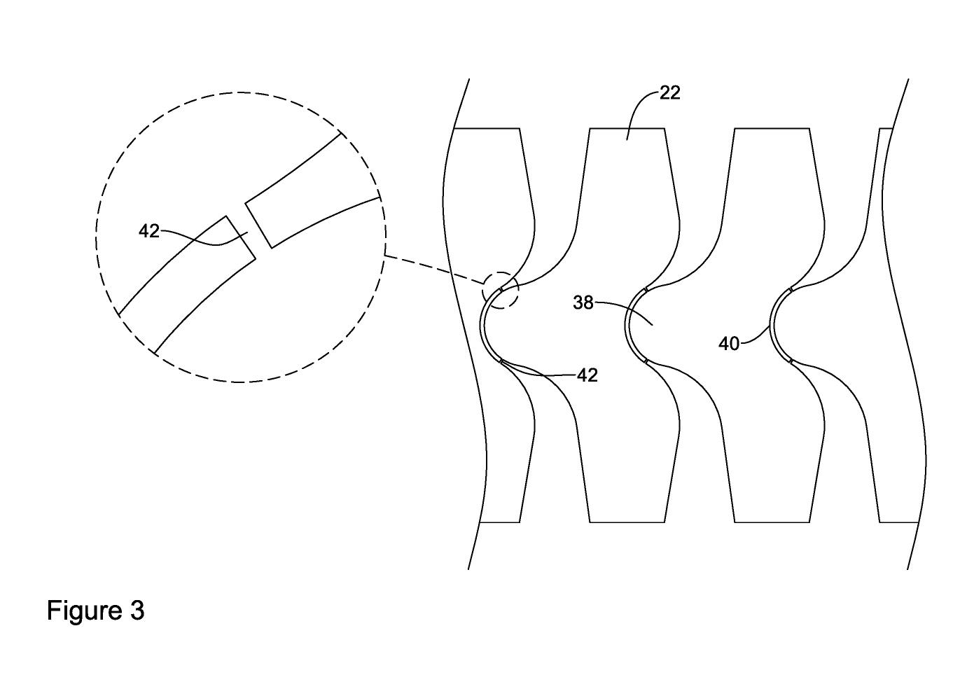

FIGS. 2-5 illustrate the tubular member 14 and some of the features thereof. For example, FIG. 2 illustrates the tubular member 14 with the ring members 22 disposed between the proximal ring member 24 and the distal ring member 26. The ring members 22 may have a desirable level of rigidity that allows the tubular member 14 to resist kinking and/or flattening. In at least some instances, each of the ring members 22 may include a knuckle region or projection 38 and a valley region or furrow 40 as shown in FIG. 3. The knuckle regions 38 and the valley regions 40 allow adjacent ring members 22 to more closely nest with one another. This may allow the tubular member 14 to have increased compression resistance, for example if the tubular member 14 is steered by actuation of a pull wire or otherwise subjected to compression. In some instances, the ring members 22 may include a single knuckle region 38 and a single valley region 40. Other instances are contemplated where each of the ring members 22 may include more than one knuckle region 38 and/or more than one valley region 40. In addition, in some instances all of the ring members 22 have the same number of knuckle regions 38 and/or the same number of valley regions 40. In other instances, some of the ring members 22 may have a differing number of knuckle regions 38 and/or a differing number of valley regions 40.

In some instances, the knuckle regions 38 may all be substantially axially aligned as depicted in FIGS. 2-3. This may allow one or more preferred bending directions to be defined in the tubular member 14 (e.g., in a direction plus or minus 90 degrees from a plane passing through all the knuckle regions 38). In other instances, the knuckle regions 38 may be distributed at different circumferential locations in different ring members 22. For example, the knuckle regions 38 of adjacent ring members 22 may be arranged in a helical pattern about the tubular member 14. A number of additional arrangements are contemplated. The valley regions 40 may be arranged in a number of different manners similar to those of the knuckle regions 38.

In at least some instances, one or more connecting members 42 may be disposed between adjacent rings as shown in FIG. 3. The connecting members 42 may be desirable for a number of reasons. For example, in some instances the tubular member 14 may be formed by a laser cutting, etching, and/or another suitable process. By cutting the tubular member 14 in a manner that “leaves behind” the connecting members 42, the tubular member 14 can be formed more efficiently and without having to mechanically arrange a plurality of discrete ring members in the desired manner. In addition, the connecting members 42 may allow the tubular member 14 to be formed in a manner that allows the spacing, arrangement, and orientation of the individual ring members 22 to be controlled.

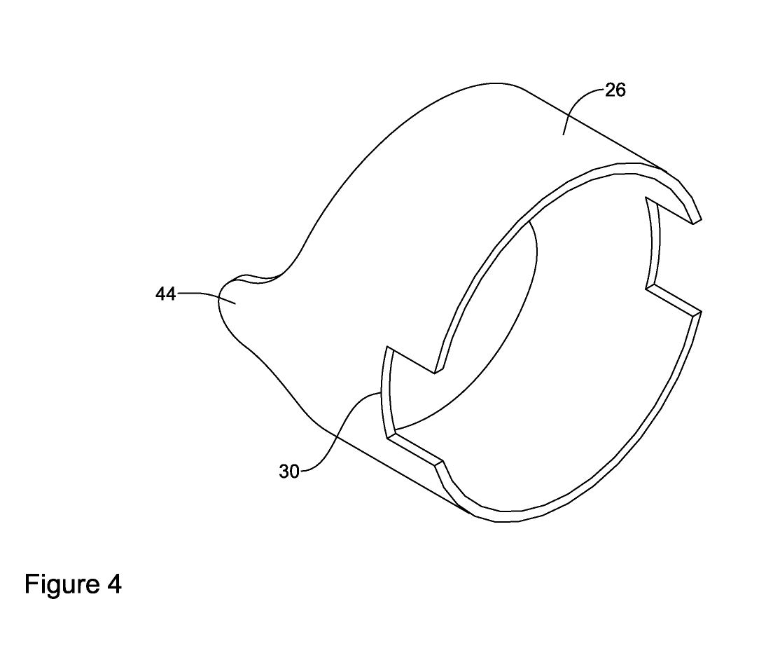

The distal ring member 26 is illustrated in FIG. 4. As indicated herein, the distal ring member 26 may include one or more cutouts 30. In this example, the distal ring member is shown having a pair of cutouts 30. However, more or fewer cutouts 30 may be utilized. In addition, in this example, the cutouts 30 have a generally squared shape. This is not intended to be limiting. Other shapes are contemplated. The squared shape of the cutouts 30 may allow a technician to more efficiently identify the distal end of the tubular member 14 during manufacturing. The distal ring member 26 may also include a knuckle region 44. The knuckle region 44 may be similar to the knuckle regions 38 of the ring members 22.

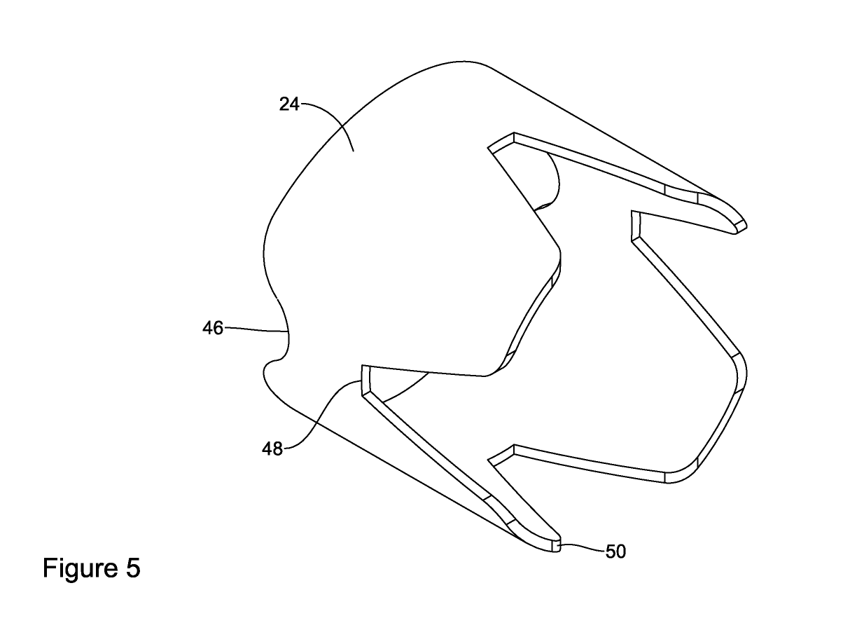

The proximal ring member 24 is illustrated in FIG. 5. The proximal ring member 24 may include a valley region 46 (e.g., which may be similar to the valley regions 40 of the ring members 22). The proximal ring member 24 may include one or more cutouts 48 defining projections 50. In this example, the cutouts 48 may be described as “V-shaped” and the projections 50 may be described as being pointed or as having an arrowhead-like shape. This is not intended to be limiting. Other shapes are contemplated. The V-shaped cutouts 48 and pointed projections 50 may allow a technician to more efficiently assembly the medical device 10, for example by allowing the proximal ring member 24 to have an increased suitable for fitting the projections 50 over the shaft member 12. For example, the projections 50 may be understood to be or otherwise include braid attachment regions of the proximal ring member 24 that are suitable for attaching the proximal ring member 24 to the braided region/support member 20 of the shaft member 12.

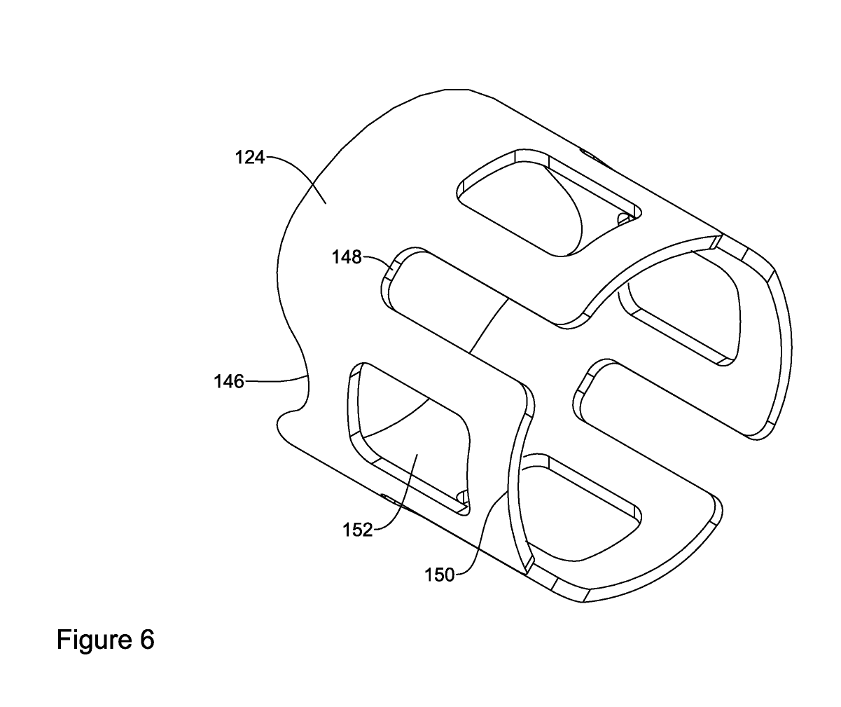

FIG. 6 illustrates an alternative proximal ring member 124. Like the proximal ring member 24, the proximal ring member 124 may include a valley region 146, cutouts 148, and projections 150. However, the shape of the cutouts 148 and the projections 150 differ to illustrate some of the alternative shapes contemplated. Like the projections 50, the projections 150 may be understood to be or otherwise include braid attachment regions of the proximal ring member 124 that are suitable for attaching the proximal ring member 124 to the braided region/support member 20 of the shaft member 12. In addition, the projections 150 of the proximal ring member 124 may include a further cutout 152. Such an arrangement may help to facilitate bonding of the proximal ring member 124 to the shaft member 12.

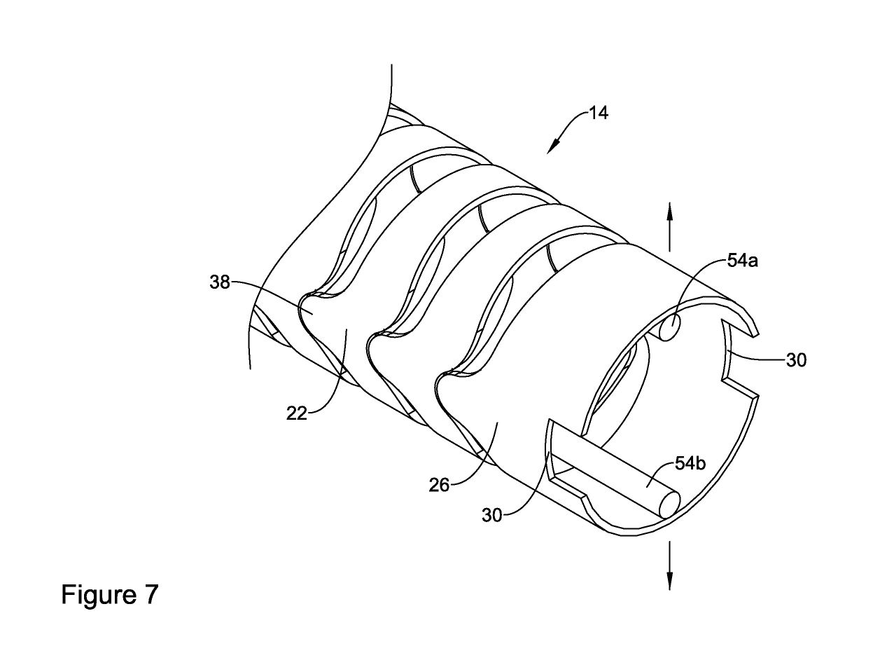

FIG. 7 illustrates a portion of tubular member 14. Here, one or more steering members 54a, 54b can be seen. In this example, the steering members 54a, 54b take the form of a pair of wires coupled to the distal ring member 26 (e.g., by a thermal bond, adhesive bond, mechanical bond, or the like) and extending along the inner surface of the tubular member 14. The steering members 54a, 54b are oriented at positions that are rotated 90 degrees from the cutouts 30 and rotated 90 degrees from the knuckle regions 38. This allows the steering members 54a, 54b to be actuated in order to steer the tubular member 14 in a pair of preferred bending directions (e.g., where the bending directions are labeled with arrows). Furthermore, the orientation of the steering members 54a, 54b with respect to the cutouts 30 and the knuckle regions 38 allows the tubular member 14 to have consistent, predictable bending that may be held within a singular plane. In some instances, two steering members 54a, 54b can be utilized. In other instances, more or fewer steering members can be utilized.

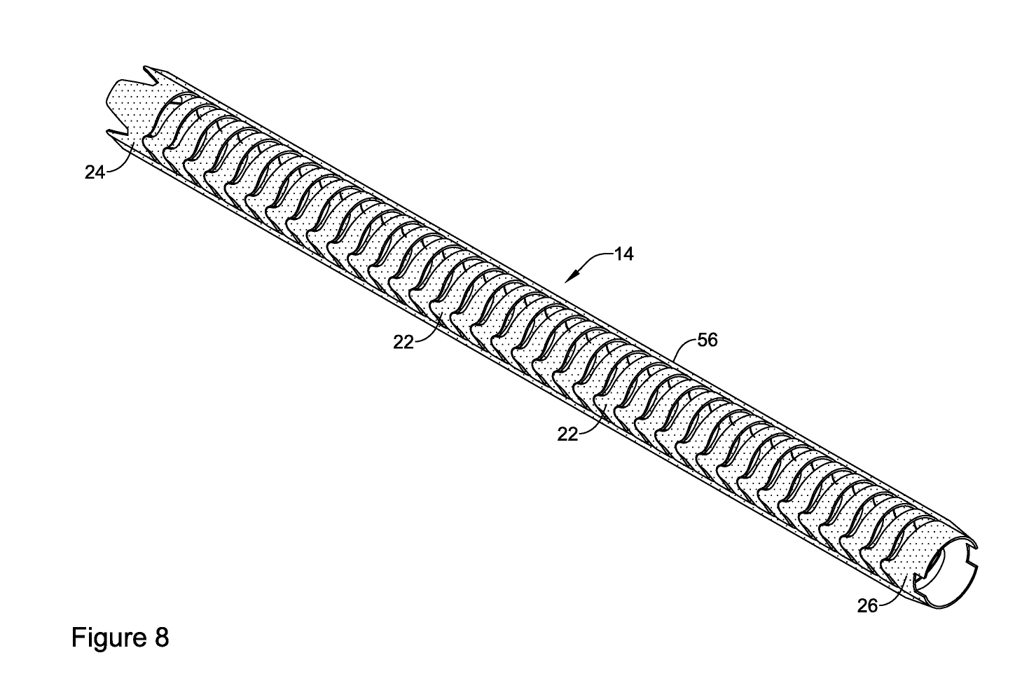

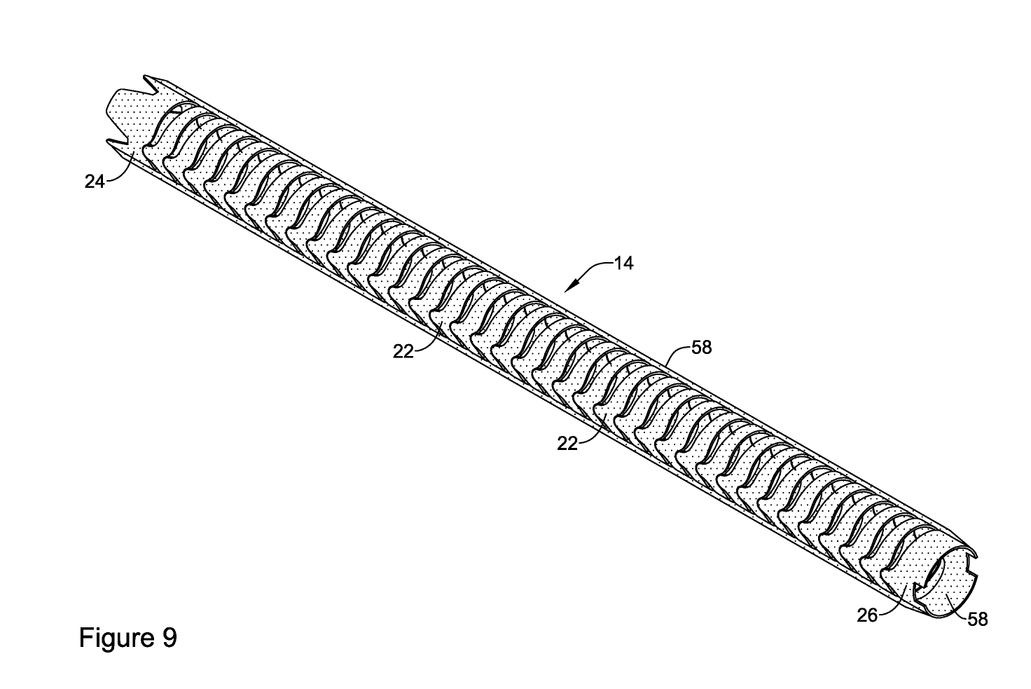

FIG. 8 illustrates that the tubular member 14 may include a polymeric member 56. In this example, the polymeric member 56 may take the form of a coating or sleeve extending along the outer surface of the tubular member 14 (e.g., along the outer surface of the ring members 22). Alternatively, FIG. 9 illustrates that the tubular member 14 may be encapsulated by a polymeric member 58 (e.g., where the polymeric member 58 extends along both the inner and outer surface of the tubular member 14 and/or along both the inner and outer surface of the ring members 22). Either way, the polymeric member 56, 58 may allow the ring members 22 (as well as the proximal ring member 24 and the distal ring member 26) of the tubular member 14 to be unified as a singular structure (e.g., the tubular member 14). For the purposes of this disclosure, references made to “the polymeric member 56, 58” may be understood to refer to either the polymeric member 56 or the polymeric member 58 in the alternative.

The manufacturing the medical device 10 may include cutting a tubular body. The tubular body may be a cylindrical tube having a lumen extending therethrough. Cutting the tubular body may include laser cutting the tubular body, etching (e.g., chemically etching) the tubular body, mechanically cutting the tubular body, machining (e.g., electron discharge machining) the tubular body, or the like, or combinations thereof. Cutting the tubular body may form or otherwise define the ring members 22 (e.g., and/or the proximal ring member 24 and/or the distal ring member 26) and the connecting members 42. The polymeric member 56, 58 may be disposed along (e.g., along the outer surface, the inner surface, or both) the ring members 22 (e.g., and/or the proximal ring member 24 and/or the distal ring member 26) to define the tubular member 14.

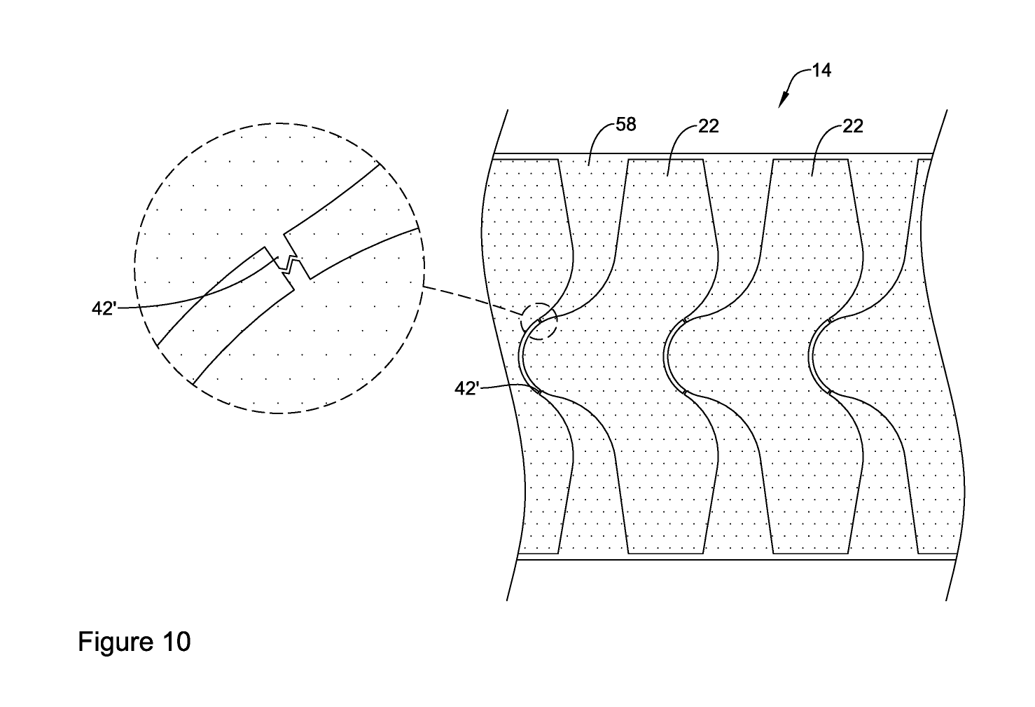

The process may also include articulating the tubular member 14 to break at least some of the connecting members 42 as depicted in FIG. 10. (e.g., where the broken connecting member is labeled in the drawing with reference number 42′). For example, the connecting members 42 may serve to aid in holding the orientation of the ring members 22 after cutting. After the polymeric member 56, 58 is coupled to the ring members 22, the connecting members 42 (which may be relatively thin and/or otherwise frangible) can be broken to allow the tubular member 14 to more freely bend at the intersections of the ring members 22, for example, by articulating or otherwise bending the tubular member 14. Articulating the tubular member 14 to break the connecting members 42 may include breaking some or all of the connecting members 42. In addition, because the polymeric member 56, 58 may be disposed along the inner surface, the outer surface, or both of the tubular member 14, the polymeric member 56, 58 may aid in preventing portions of the connecting members 42 from becoming separated from the tubular member 14. In other words, the polymeric member 56, 58, may contain and hold any loose fragments that might otherwise be freed when the connecting members 42 are broken.

In order to sufficiently break the connecting members 42, the tubular member 14 may be manually articulated (e.g., bent) by a user one or more times. In some instances, the tubular member 14 may be articulated by bending the tubular member 14 to angles greater than or equal to about 30 degrees, or to angles greater than or equal to about 60 degrees, or to angles greater than or equal to about 90 degrees, or to angles greater than or equal to about 135 degrees. The process may include bending the tubular member 14 in a first direction (e.g., to an angle greater than or equal to about 90 degrees) and then bending the tubular member 14 in an opposite direction (e.g., to an angle greater than or equal to about 90 degrees in the opposite direction). The process may be repeated. The connecting members 42 may have a reduced thickness relative to a thickens of the ring members 22 such that the connecting members 42 can break at a lower threshold (e.g., torsional force, etc.) as compared to the ring members 22. In an example, the connecting members 42 may be described as being frangible. For example, the connecting members 42 may be designed to break at a lower force that the ring members 22. In some instances, a level of audible feedback may communicate to the user that the connecting members 42 are broken. In other instances, visual inspection may reveal the successful breaking of the connecting members 42.

In order to further facilitate breaking the connecting members 42, the connecting members 42 may include one or more structural configurations that may facilitate breaking. For example, one or more regions of the connecting members 42 may include a beveled or angled surface along one or more edges. In some of these and in other instances, one or more of the connecting members 42 may include a thinned region (e.g., near a central portion thereof). These are just examples. Other forms and configurations are contemplated.

Alternatively or additionally, the process may include coupling the tip member 16 to the tubular member 14 (e.g., coupling the tip member 16 to the distal ring member 26). Alternatively or additionally, the process may include coupling the tubular member 14 to the shaft member 12 (e.g., coupling the tip member 16 to the proximal ring member 24, for example by securing the braid attachment region/projection 50 of the proximal ring member 24 to the braid/support member 20 of the shaft member 12). Alternatively or additionally, the process may include coupling one or more steering members 54a, 54b to the tubular member 14.

The materials that can be used for the various components of the medical device 10 and the various components thereof may include those commonly associated with medical devices. For simplicity purposes, the following discussion makes reference to the tubular member 14 and other components of the medical device 10. However, this is not intended to limit the devices and methods described herein, as the discussion may be applied to other similar tubular members and/or components of tubular members or devices disclosed herein.

The tubular member 14 and/or other components of the medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (MRI) compatibility is imparted into the medical device 10. For example, the medical device 10, or portions thereof, may be made of a material that does not substantially distort the image and create substantial artifacts (e.g., gaps in the image). Certain ferromagnetic materials, for example, may not be suitable because they may create artifacts in an MRI image. The medical device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method for manufacturing a medical device, the method comprising:
cutting a tubular body into a plurality of ring members, wherein the plurality of ring members includes a first ring member and a second ring member, wherein a connecting member extends between the first ring member and the second ring member;
disposing a polymeric member along the plurality of ring members to define a tubular member; and
articulating the tubular member to break the connecting member.

2. The method of claim 1, wherein cutting a tubular body into a plurality of ring members includes laser cutting.

3. The method of claim 1, wherein cutting a tubular body into a plurality of ring members includes etching.

4. The method of claim 1, wherein disposing a polymeric member along the plurality of ring members includes disposing the polymeric member along at least a portion of an outer surface of the plurality of ring members.

5. The method of claim 1, wherein disposing a polymeric member along the plurality of ring members includes encapsulating at least a portion of the plurality of ring members within the polymeric member.

6. The method of claim 1, wherein a second connecting member extends between the first ring member and the second ring member, and wherein articulating the tubular member to break the connecting member includes breaking the second connecting member.

7. The method of claim 1, further comprising attaching the tubular member to a shaft member.

8. The method of claim 7, wherein the shaft member includes a braided region, wherein the tubular member includes a proximal ring member with a braid attachment region, and wherein attaching the tubular member to a shaft member includes securing the braid attachment region of the proximal ring member to the braided region.

9. The method of claim 1, further comprising attaching a steering member to the tubular member.

10. The method of claim 9, wherein attaching a steering member to the tubular member includes disposing the steering member along an inner surface of the tubular member and securing the steering member to a distal end region of the tubular member.

11. A medical device, comprising:
a tubular member including a plurality of ring members, wherein the plurality of ring members includes a distal ring member, a first body region ring member, a second body region ring member, and a proximal ring member;
a frangible connecting member extending between the first body region ring member and the second body region ring member;
a polymeric member disposed along the tubular member; and
a shaft member coupled to the proximal ring member.

12. The medical device of claim 11, further comprising a second frangible connecting member extending between the first body region ring member and the second body region ring member.

13. The medical device of claim 11, wherein the first body region ring member has a knuckle region and wherein the knuckle region is nested with a valley region of the second body region ring member.

14. The medical device of claim 11, wherein the shaft member includes a braid and wherein the proximal ring member includes a braid attachment region coupled to the braid.

15. The medical device of claim 11, further comprising a steering member coupled to the tubular member.

16. The medical device of claim 15, wherein the steering member extends along an inner surface of the tubular member and is secured to the distal ring member.

17. The medical device of claim 11, wherein the polymeric member includes a sleeve disposed along an outer surface of the tubular member.

18. The medical device of claim 11, wherein the polymeric member encapsulates the tubular member.

19. A method for manufacturing a medical device, the method comprising:
encapsulating a plurality of interconnected ring members in a polymeric member to form a tubular member, the plurality of interconnected ring members including a distal ring member, a first body region ring member, a second body region ring member, a frangible connecting member extending between the first body region ring member and the second body region ring member, and a proximal ring member;
articulating the tubular member to break the frangible connecting member; and
coupling the proximal ring member to a shaft member.

20. The method of claim 19, wherein the plurality of interconnected ring members includes a third body region ring member and a second frangible connecting member extending between the second body region ring member and the third body region ring member, and wherein articulating the tubular member to break the frangible connecting member breaks the second frangible connecting member.
